(19) United States
(12) Patent Application Publication
(10) Pub. No.: US 2013/0079889 A1
(43) Pub. Date: Mar. 28, 2013

(54) Irradiated Cortical Bone Sheet Allografts and Method of Forming Irradiated Cortical Bone Sheet Allografts

(71) Applicant: Deborah Marie Spillman, Denver, CO (US)

(72) Inventor: Deborah Marie Spillman, Denver, CO (US)

(21) Appl. No.: 13/629,060

(22) Filed: Sep. 27, 2012

Related U.S. Application Data
(60) Provisional application No. 61/539,781, filed on Sep. 27, 2011.

(51) Int. Cl. A61F 2/28

(52) U.S. Cl. 623/23.63

(57) ABSTRACT

There is disclosed an irradiated cortical bone sheet allograft. In an embodiment, the allograft includes a unitary sheet of at least partially demineralized, irradiated cortical bone having a thickness, a width, and a length. The thickness of the unitary sheet of irradiated cortical bone is less the width and the length. In another embodiment, a method of forming the allograft includes obtaining a natural bone from a donor different than a recipient. The natural bone contains a layer of a cortical bone. The method includes cleaning the natural bone to produce a unitary sheet of cortical bone. The method includes at least partially demineralizing the unitary sheet of cortical bone. The method includes freezing the unitary into a frozen state within a sealed package. The method includes irradiating the unitary sheet in the frozen state within the sealed package to sterilize the cortical bone and produce the irradiated cortical bone sheet for implantation in the recipient other than the donor. Other embodiments are also disclosed.

### REFERENCE TO PENDING PRIOR PATENT APPLICATION

This application claims the benefit under 35 U.S.C. 119 (e) of U.S. Provisional Patent Application No. 61/539,781, filed Sep. 27, 2011 by Deborah Marie Spillman for “IRRADIATED CORTICAL BONE SHEET ALLOGRAFTS AND METHOD OF FORMING IRRADIATED CORTICAL BONE SHEET ALLOGRAFTS,” which patent application is hereby incorporated herein by reference.

### BACKGROUND

Generally, allograft bone materials are demineralized and either lyophilized or dehydrated. The allograft bone materials may be processed with a demineralization process, a dehydration process, or both. These processes change the grafts at a molecular level.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In an embodiment, there is provided an irradiated cortical bone sheet allograft, the allograft comprising a unitary sheet of at least partially demineralized, irradiated cortical bone having a given thickness, a given width, and a given length, wherein the given thickness of the unitary sheet of at least partially demineralized, irradiated cortical bone is less than each one of the given width and the given length, and the unitary sheet of at least partially demineralized, irradiated cortical bone being packaged in a sterile environment.

In another embodiment, there is provided an irradiated cortical bone sheet allograft, the allograft comprising a unitary sheet of at least partially demineralized, irradiated cortical bone packaged with a liquid carrier in a sterile environment at room temperature.

In yet another embodiment, there is provided a method of forming an irradiated cortical bone sheet allograft, the method comprising obtaining a natural bone from a donor different than a recipient, the natural bone containing a layer of a cortical bone; cleaning the natural bone to produce a unitary sheet of cortical bone; a least partially demineralizing the unitary sheet of cortical bone; freezing the unitary sheet of cortical bone into a frozen state within a sealed package; and irradiating the unitary sheet of cortical bone in the frozen state within the sealed package to sterilize the unitary sheet of cortical bone and producing the irradiated cortical bone sheet for implantation in the recipient other than the donor.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIGS. 1A and 1B illustrate two embodiments of packaged irradiated cortical bone sheet allograft products; and

FIGS. 2-19 illustrate various sized embodiments of an irradiated cortical bone sheet allograft.

### DETAILED DESCRIPTION

Embodiments are described more fully below in sufficient detail to enable those skilled in the art to practice the allograft and method. However, embodiments may be implemented in many different forms and should not be construed as being limited to the embodiments set forth herein. The following detailed description is, therefore, not to be taken in a limiting sense.

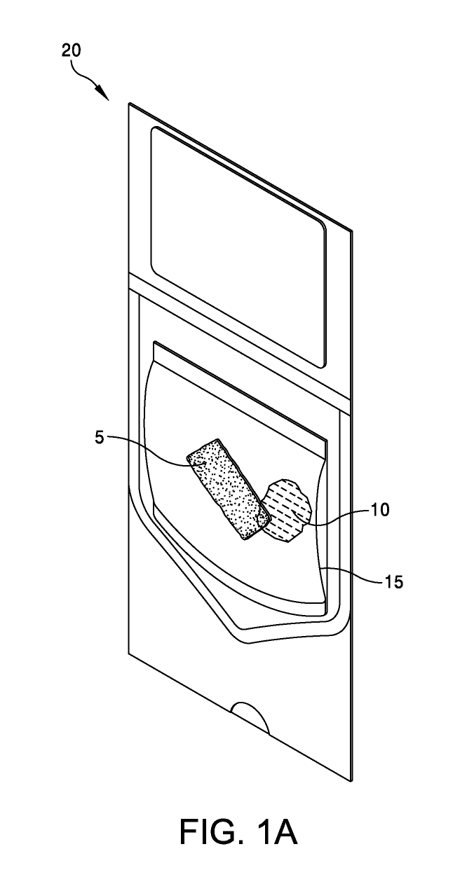

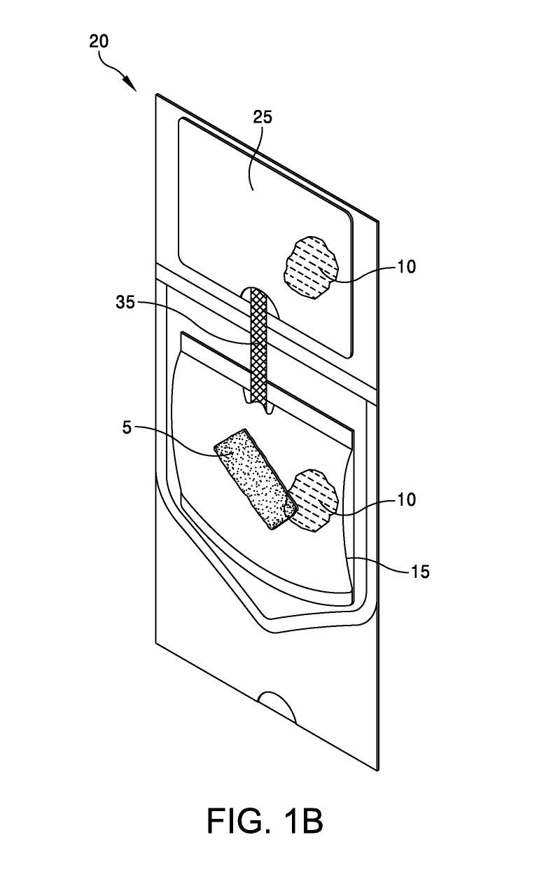

In an embodiment, and with references to FIGS. 1A, 1B, and 2-19, an allograft bone sheet is provided from a thinly sliced sheet 5. The allograft bone sheet may be presented at the time of surgery in a natural state. In one embodiment, the allograft bone sheet 5 may be provided for surgery at an ambient air temperature rather than in a frozen configuration. The allograft bone sheet 5 is not manipulated, ground, crushed, nor matted out. Nothing is added to the sheet graft. The allograft bone sheet 5 may be presented more similar to a fresh or fresh frozen graft. Irradiation preserves the graft to allow storage in an ambient air environment rather than in a controlled temperature environment. In one embodiment, the allograft bone sheet 5 is fully mineralized when packaged and subsequently at least partially demineralized prior to implantation. In another embodiment, the allografts bone sheet 5 is at least partially demineralized when packaged.

The allograft bone sheet may be produced more cost effectively by eliminating or reducing the cost of demineralization, dehydration, or both demineralization and dehydration. This allograft eliminates the need for a surgeon to harvest a sheet of bone from the patient to produce an autograft bone sheet.

In one embodiment, donor bone material is processed into a desired size of an allograft bone sheet 5. For example, a bone may be thinly sliced to produce one or more thinly sliced sheets of cortical bone. This bone may be a long bone or an iliac bone. Examples of long bones include, but are not limited to, the femur, tibia, fibula, humerus, radius, and ulna.

Next, the allograft bone sheet 5 may be packaged in a liquid carrier 10 within a plastic pouch 15. In an embodiment, the allograft bone sheet 5 is hydrated with, and packaged within, sterile water as the liquid carrier 10. FIGS. 1A and 1B illustrate an exemplary embodiment of a packaged allograft bone sheet 20.

The packaged allograft bone sheet 5 may be frozen after packaging and prior to irradiation. In one embodiment, the packaged allograft bone sheet may be frozen to approximately −70 degrees Celsius.

The frozen, packaged allograft bone sheet 5 may be irradiated while frozen. An adequate irradiation procedure is used for sterilization of the allograft bone sheet 5. A Cobalt 60 source may be used to apply irradiation in a range of 2.5 to 3.8 mRads (25 to 38 kGy) of irradiation.

In an embodiment, the packaged allograft product 20 may be allowed to thaw after irradiation to room temperature. The packaged allograft product 20 may be stored at room temperature after irradiation until use by a surgeon. In the various embodiments disclosed herein, the irradiated cortical bone sheet allograft 5 is not demineralized or dehydrated prior to use at a surgical site. In some embodiments, the irradiated cortical bone sheet allograft is at least partially demineralized prior to irradiation. This demineralization may occur within, or apart from, the plastic pouch 15 or other packaging.

With reference to FIG. 1B, and in an embodiment, a reservoir 25 containing an organic acid 30 may be provided in packaged allograft product 20. The organic acid 30 may include, but is not limited to, citric acid, nitric acid, or hydrochloric acid. At an appropriate time, a dentist, surgeon or other end user may cause the organic acid 30 to bathe the allograft bone sheet 5. In one embodiment, a communication channel 35 may be opened to direct the organic acid into the pouch 15 and mix with liquid carrier 10. In order to obtain a desired about of demineralization, the timing of the application of the organic acid, as well as the concentration of the organic acid, may be regulated. The volume of the organic acid 30 and the volume of the liquid carrier 10 may be selected to cause at least partial demineralization to a desired flexibility of the allograft bone sheet 5 prior to implantation. In other embodiments (not shown), different types of packaging, including, but not limited to, a releasable organic acid-containing pouch may be provided to selectively apply the organic acid to the allograft bone sheet when the pouch is broken and may not require a communication channel.

The amount of mineral removed from the bone may be adjusted to create the desired amount of flexibility. This demineralization conventionally uses an organic acid such as hydrochloric, nitric, or citric acid. In an embodiment, the demineralization solution comprises 0.1 to 1.0 N HCl, most preferably 0.3 N HCl. Once the sheet has been machined and partially demineralized, it may be stored prior to insertion.

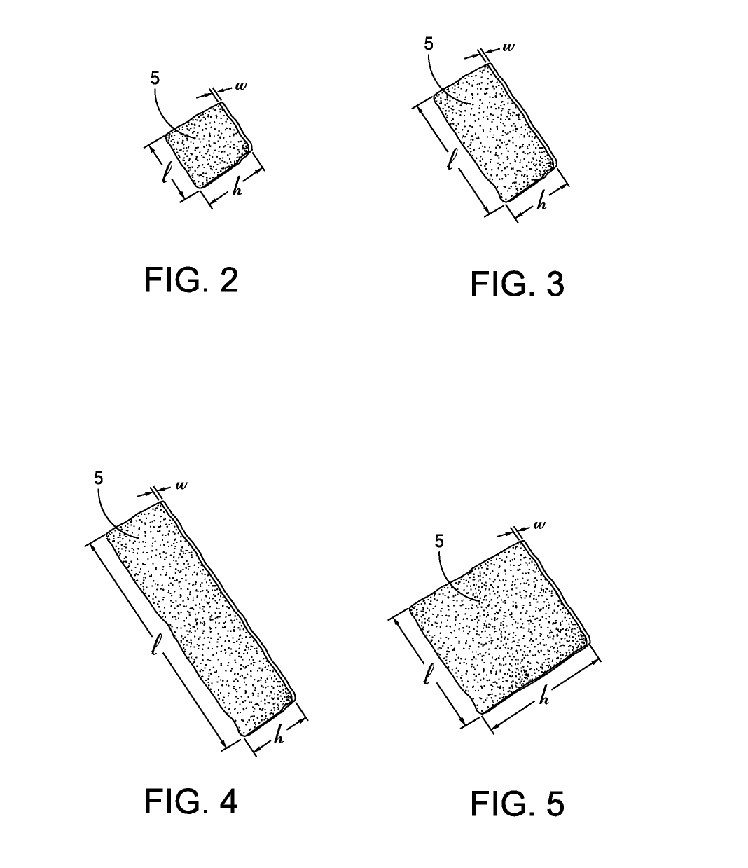

FIGS. 2-19 illustrate various exemplary sizes of allograft bone sheets 5. These sheets include a thickness smaller than with respect to a length and a width. The thickness of the allograft bone sheets 5 includes cortical bone. In an embodiment, the allograft bone sheets 5 each consist of, and are limited to, cortical bone. In one embodiment, the allograft bone sheets each comprise, but are not limited to, cortical bone.

In various embodiments, irradiated cortical bone sheet allografts 5 may be sized from about 0.5 mm to 1.5 mm in thickness, 10 mm to 40 mm in width, and 10 mm to 40 mm in length.

FIG. 2 illustrates an irradiated cortical bone sheet allograft 5 having dimensions with a thickness of about 0.5 mm by a width of about 10 mm length by a length of about 10 mm (0.5 mm×10 mm ×10 mm.)

FIG. 3 illustrates an irradiated cortical bone sheet allograft 5 having dimensions with a thickness of about 0.5 mm by a width of about 10 mm length by a length of about 10 mm (0.5 mm×10 mm×20 mm.)

FIG. 4 illustrates an irradiated cortical bone sheet allograft 5 having dimensions with a thickness of about 0.5 mm by a width of about 10 mm length by a length of about 40 mm (0.5 mm×10 mm×40 mm.)

FIG. 5 illustrates an irradiated cortical bone sheet allograft 5 having dimensions with a thickness of about 0.5 mm by a width of about 20 mm length by a length of about 20 mm (0.5 mm×20 mm×20 mm.)

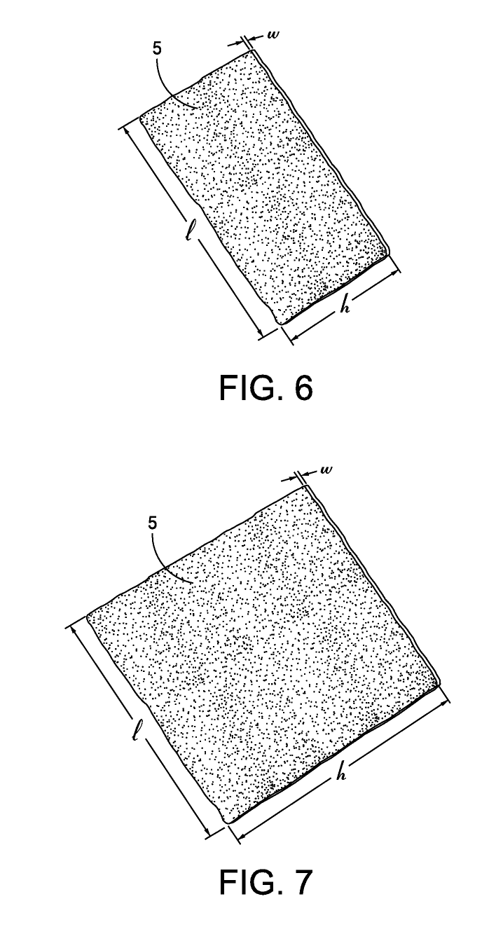

FIG. 6 illustrates an irradiated cortical bone sheet allograft 5 having dimensions with a thickness of about 0.5 mm by a width of about 20 mm length by a length of about 40 mm (0.5 mm×20 mm×40 mm.)

FIG. 7 illustrates an irradiated cortical bone sheet allograft 5 having dimensions with a thickness of about 0.5 mm by a width of about 40 mm length by a length of about 40 mm (0.5 mm×40 mm×40 mm.)

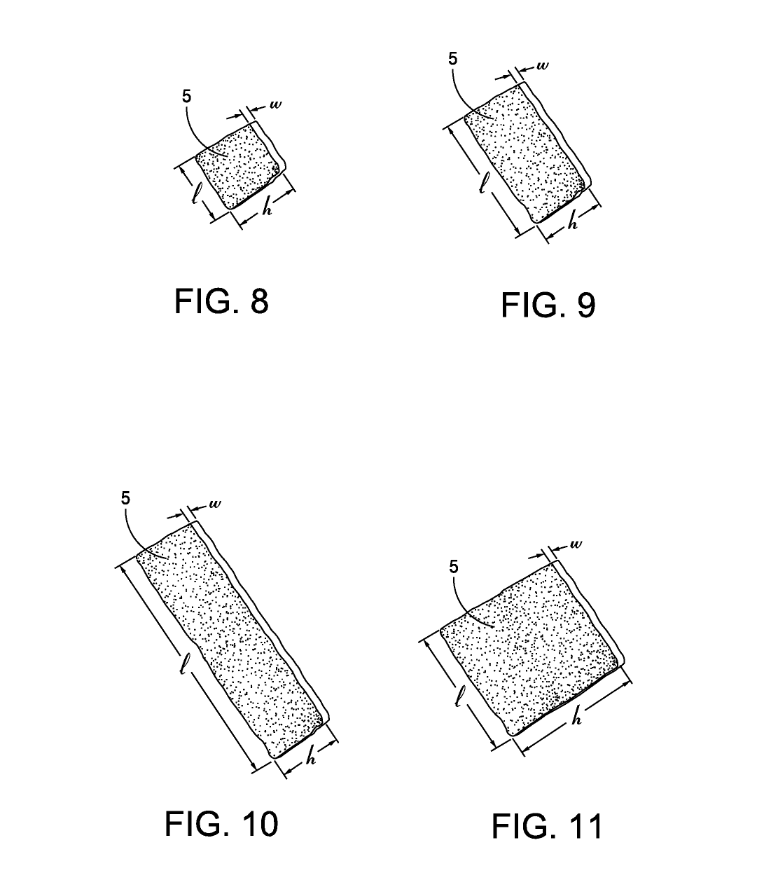

FIG. 8 illustrates an irradiated cortical bone sheet allograft 5 having dimensions with a thickness of about 1 mm by a width of about 10 mm length by a length of about 10 mm (1 mm×10 mm×10 mm.)

FIG. 9 illustrates an irradiated cortical bone sheet allograft 5 having dimensions with a thickness of about 1 mm by a width of about 10 mm length by a length of about 20 mm (1 mm×10 mm×20 mm.)

FIG. 10 illustrates an irradiated cortical bone sheet allograft 5 having dimensions with a thickness of about 1 mm by a width of about 10 mm length by a length of about 40 mm (1 mm×10 mm×40 mm.)

FIG. 11 illustrates an irradiated cortical bone sheet allograft 5 having dimensions with a thickness of about 1 mm by a width of about 20 mm length by a length of about 20 mm (1 mm×20 mm×20 mm.)

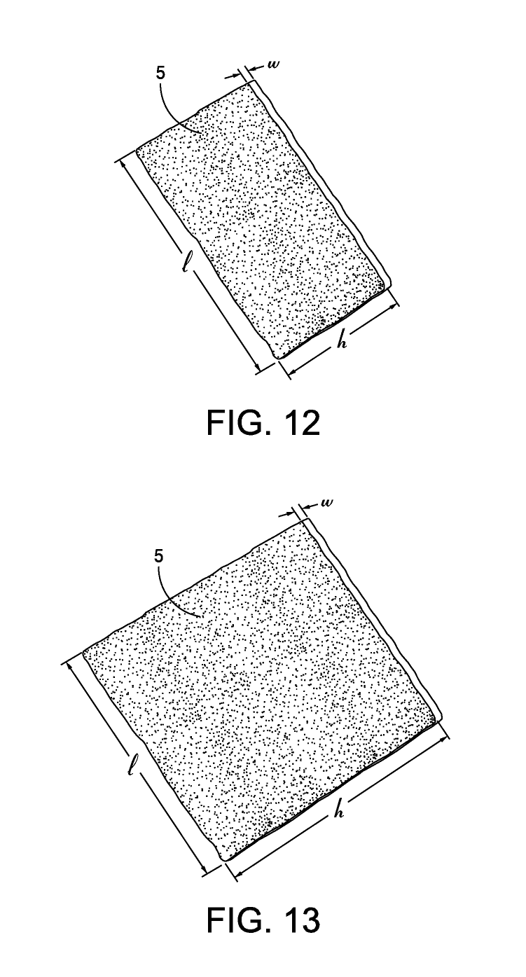

FIG. 12 illustrates an irradiated cortical bone sheet allograft 5 having dimensions with a thickness of about 1 mm by a width of about 20 mm length by a length of about 10 mm (1 mm×20 mm×40 mm.)

FIG. 13 illustrates an irradiated cortical bone sheet allograft 5 having dimensions with a thickness of about 1 mm by a width of about 40 mm length by a length of about 40 mm (1 mm×40 mm×40 mm.)

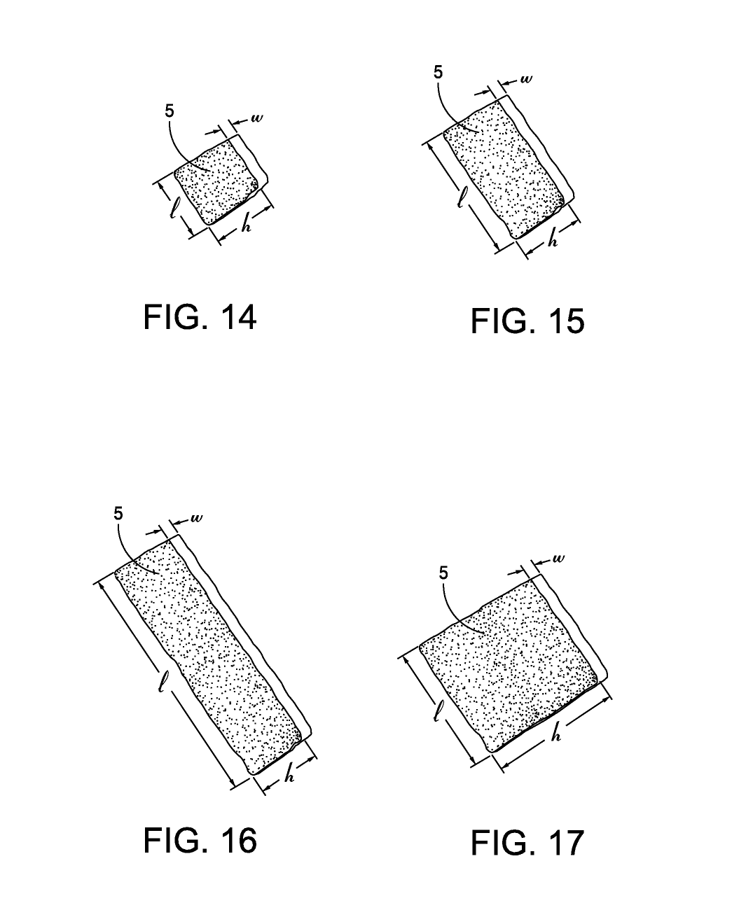

FIG. 14 illustrates an irradiated cortical bone sheet allograft 5 having dimensions with a thickness of about 1.5 mm by a width of about 10 mm length by a length of about 10 mm (1.5 mm×10 mm×10 mm.)

FIG. 13 illustrates an irradiated cortical bone sheet allograft 5 having dimensions with a thickness of about 1.5 mm by a width of about 10 mm length by a length of about 20 mm (1.5 mm×10 mm×20 mm.)

FIG. 14 illustrates an irradiated cortical bone sheet allograft 5 having dimensions with a thickness of about 1.5 mm by a width of about 10 mm length by a length of about 40 mm (1.5 mm×10 mm×40 mm.)

FIG. 17 illustrates an irradiated cortical bone sheet allograft 5 having dimensions with a thickness of about 1.5 mm by a width of about 20 mm length by a length of about 20 mm (1.5 mm×20 mm×20 mm.)

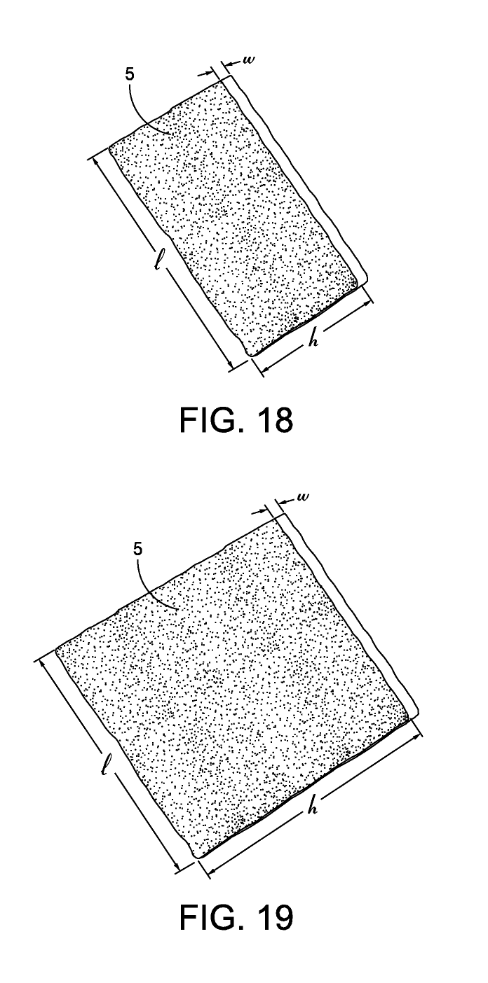

FIG. 18 illustrates an irradiated cortical bone sheet allograft 5 having dimensions with a thickness of about 1.5 mm by a width of about 20 mm length by a length of about 40 mm (1.5 mm×20 mm×40 mm.)

FIG. 19 illustrates an irradiated cortical bone sheet allograft 5 having dimensions with a thickness of about 1.5 mm by a width of about 40 mm length by a length of about 40 mm (1.5 mm×40 mm×40 mm.)

The irradiated cortical bone sheet allograft 5 is designed as a barrier, scaffolding matrix, or both, when utilized in a guided bone regeneration surgical application. For example, within a surgical site in the body, a surgeon may place an irradiated cortical bone sheet allograft 5 together with other osteo-integration material at a graft site. The irradiated cortical bone sheet allograft will guide the osteo-integration material by slowing the regeneration process of bone, and stop the ingrowth of soft tissue into the graft site. In one embodiment, the irradiated cortical bone sheet allograft may be formed in a tunnel shape to create a matrix, and the irradiated cortical bone sheet allograft used as a barrier to stop soft tissue growth into the graft site.

In one embodiment, the irradiated cortical bone sheet allograft 5 may be positioned at a surgical site to create a package into which platelet rich plasma (PRP) is positioned within the barrier formed by the irradiated cortical bone sheet allograft 5.

In another embodiment, the irradiated cortical bone sheet allograft 5 may be positioned at a surgical site near a native bone that has been de-corticalized. Removal of the cortical portion of the native bone causes blood and other substances to well up into an enclosed region formed by the irradiated cortical bone sheet allograft 5. This may provide better soft tissue healing at the surgical site.

A fully demineralized sheet of allograft bone may resorb at a surgical site faster than a partially demineralized sheet of bone (i.e., the irradiated cortical bone sheet allograft 5.) For some surgical applications, slower resorbing of the bone is a positive aspect to allow enough time for ingrowth of other implanted particulate material. The partially demineralized physical properties and mechanical aspects of the irradiated cortical bone sheet allograft are also very different from those of a fully demineralized sheet of allograft bone.

As the sheets are not fully demineralized, there is little or no shrinkage and the initially cut sheets provide a more predictable dimensional tailoring of the final bone sheet. The bone sheets are described hereinabove as generally square or rectangular faces forming a cuboid. However, other three dimensional “sheets” may be constructed with a substantially thin thickness in comparison to larger widths and lengths.

Irradiation preserves the irradiated cortical bone sheet allograft 5. This allows storage of the irradiated cortical bone sheet allograft 5 in ambient air (within a sealed pouch 15) rather than in a controlled temperature environment. The irradiated cortical bone sheet allograft 5 may be produced more cost effectively than other types of cortical, cortical cancellous, or cancellous bone implants. The irradiated cortical bone sheet allograft are less expensive to produce because of the elimination or reduction of the cost of demineralization, dehydration, or a combination of each of these processes. The irradiated cortical bone sheet allograft 5 also eliminates the cost of a surgeon having to harvest a sheet of bone (i.e., an irradiated cortical bone sheet allograft) from a patient. The advantages of allografts include ready availability, elimination of the need for a patient donor site, reduced anesthesia and surgical time, decreased blood loss, and fewer complications. The disadvantages of allografts are primarily associated with the antigenicity of tissues harvested from another individual. Irradiated cancellous bone (Rocky Mountain Tissue Bank, Denver, Colo., USA) has also been used as substitute graft material to autogenous bone. Allogeneic bone behaves similar to autologous bone in terms of creeping substitution and osseointegration of implants.

Irradiation also releases growth factors for soft tissue formation.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. An irradiated cortical bone sheet allograft product, the allograft product comprising:
a unitary sheet of at least partially demineralized, irradiated cortical bone having a given thickness, a given width, and a given length, wherein the given thickness of the unitary sheet of at least partially demineralized, irradiated cortical bone is less than each one of the given width and the given length, and the unitary sheet of at least partially demineralized, irradiated cortical bone being packaged in a sterile environment.

2. The allograft product of claim 1, wherein the unitary sheet comprises an integral sheet of cortical bone from a donor different than a recipient.

3. The allograft product of claim 1, wherein the given thickness is from about 0.5 mm to about 1.5 mm, the given length is from about 10 mm to about 40 mm, and the given width is about 10 mm to about 40 mm.

4. The allograft product of claim 1, wherein the sterile environment is a sealed package.

5. The allograft product of claim 4, wherein the sealed package is configured for storage at room temperature after sterilization of the irradiated cortical bone.

6. The allograft product of claim 4, wherein the sealed package comprises a reservoir containing an organic acid.

7. The allograft product of claim 6, wherein the organic acid is citric acid.

8. The allograft product of claim 6, wherein the organic acid is nitric acid.

9. The allograft product of claim 6, wherein the organic acid is hydrochloric acid.

10. The allograft product of claim 1, wherein the sterile environment is a sealed package configured for freezing and irradiation for sterilization of the unitary sheet of the cortical bone.

11. The allograft product of claim 1, wherein the cortical bone is fully demineralized.

12. The allograft product of claim 1, further comprising sterile water disposed in the sterile environment.

13. The allograft product of claim 1, wherein the unitary sheet consists of cortical bone.

14. An irradiated cortical bone sheet allograft product, the allograft comprising:
a unitary sheet of at least partially demineralized, irradiated cortical bone packaged with a liquid carrier in a sterile environment at room temperature.

15. The allograft product of claim 14, wherein the unitary sheet comprises an integral sheet of cortical bone from a donor different than a recipient.

16. The allograft product of claim 14, wherein the sterile environment is a sealed package.

17. The allograft product of claim 16, wherein the sealed package configured for storage at room temperature after sterilization of the irradiated cortical bone.

18. The allograft product of claim 17, wherein the sterile environment is a sealed package configured for freeing and irradiation for sterilization of the unitary sheet of the cortical bone.

19. The allograft product of claim 14, wherein the cortical bone is fully demineralized.

20. The allograft product of claim 14, wherein the unitary sheet consists of at least partially demineralized cortical bone.

21. A method of forming an irradiated cortical bone sheet allograft, the method comprising:
obtaining a natural bone from a donor different than a recipient, the natural bone containing a layer of a cortical bone;
cleaning the natural bone to produce a unitary sheet of cortical bone;
at least partially demineralizing the unitary sheet of cortical bone;
freezing the unitary sheet of cortical bone into a frozen state within a sealed package; and
irradiating the unitary sheet of cortical bone in the frozen state within the sealed package to sterilize the unitary sheet of cortical bone and producing the irradiated cortical bone sheet for implantation in the recipient other than the donor.

22. The method of claim 21, wherein the step of shaping the natural bone further comprises sizing the unitary sheet of cortical bone to a given thickness, a given width, and a given length, wherein the given thickness is less than each one of the given width and the given length.

23. The method of claim 21, further comprising allowing the unitary sheet of cortical bone to thaw to room temperature within the sealed package after the step of irradiating the unitary sheet of cortical bone.

24. The method of claim 21, further comprising fully demineralizing the unitary sheet of cortical bone.

25. The method of claim 21, further comprising hydrating the unitary sheet of cortical bone with sterile water in the sealed package prior to the step of freezing the unitary sheet of cortical bone.

26. The method of claim 21, wherein the step of at least partially demineralizing the unitary sheet of cortical bone precedes the steps of freezing the unitary sheet of cortical bone and irradiating the sheet of cortical bone.

27. The method of claim 21, wherein the step of at least partially demineralizing the unitary sheet of cortical bone follows the steps of freezing the unitary sheet of cortical bone and irradiating the sheet of cortical bone.

28. The method of claim 21, wherein the step of at least partially demineralizing the unitary sheet occurs within the sealed package.

29. The method of claim 21, wherein the sealed package comprises a reservoir containing an organic acid, and wherein the step of at least partially demineralizing the unitary sheet comprises transferring the organic acid from the reservoir to the unitary sheet.

30. The method of claim 24, wherein the organic acid is citric acid.

31. The method of claim 24, wherein the organic acid is nitric acid.

32. The method of claim 24, wherein the organic acid is hydrochloric acid.
